(19) United States
(12) Patent Application Publication
(10) Pub. No.: US 2025/0345474 A1
(43) Pub. Date: Nov. 13, 2025

(54) METHOD FOR THE CHEMICAL DISINFECTION OF A REVERSE OSMOSIS SYSTEM AND REVERSE OSMOSIS SYSTEM

(71) Applicant: B. Braun Avitum AG, Melsungen (DE)

(72) Inventors: Nino Lieb, Melsungen (DE); Philipp Odernheimer, Melsungen (DE)

(21) Appl. No.: 19/199,908

(22) Filed: May 6, 2025

(30) Foreign Application Priority Data
May 7, 2024 (DE) 10 2024 112 730.9

(51) Int. Cl. A61L 2/18; C02F 1/44

(52) U.S. Cl. CPC A61L 2/18; C02F 1/441; A61L 2202/14; C02F 2303/04

(57) ABSTRACT

A method is used for the chemical disinfection of a reverse osmosis system. The system has a supply tank, a ring line for connecting consumers, at least one membrane module, a feed line to the membrane module, at least one pressure pump for pumping liquid into the membrane module and a disinfectant container. Disinfectant is conveyed from the disinfectant container into the supply tank. The includes comprises the steps of setting a target concentration of the disinfectant, determining the amount of disinfectant required based on the target concentration of the disinfectant, and discharging the amount of disinfectant required for the target concentration from the disinfectant container into the supply tank through a discharge device.

### CROSS-REFERENCE TO RELATED APPLICATIONS

This application claims priority under 35 U.S.C. § 119 to German Application No. 10 2024 112 730.9, filed on May 7, 2024, the content of which is incorporated by reference herein in its entirety.

### BACKGROUND

In the chemical disinfection of reverse osmosis systems for the production of permeate for dialysis therapy, the disinfectant quantities usually have to be calculated manually by the user and then filled into the system. In addition, the systems are not able to regulate the disinfectant quantities during operation.

Currently, chemical disinfection involves disconnecting all dialysis machines from the ring main and optionally switching off the fresh water supply from a certain process step. This means that only the reverse osmosis system and the ring main can be chemically disinfected. The disinfectant is either poured into the tank of the reverse osmosis system by a person or automatically sucked in by the device.

DE 195 38 818 A1 discloses a system for supplying a dialysis station with dialysis water, with a ring line to which the dialysis station is connected, and with a reverse osmosis module for producing the dialysis water, wherein the permeate produced by the reverse osmosis module from a raw water connection can be introduced into the ring line as dialysis water via a flow valve. An inoculant line for supplying chemical cleaning agents is connected to the ring line, whereby a dosing pump is arranged in the inoculant line.

DE 10 2006 026 107 B3 discloses a device for disinfecting a reverse osmosis system with a pump which conveys the chemical disinfectant taken from an atmospherically ventilated supply tank via a suction line into the line system of the reverse osmosis system. The reverse osmosis system has a suction chamber inserted into the suction line with an upper connection to the pump and a lower connection to the supply tank and with an aeration line connected to the upper part, which is provided with a shut-off device which is closed in the operating state of the disinfectant supply and is open during the other operating states of the reverse osmosis system.

The disadvantage of known methods is that the dialysis machines in the center are also supplied centrally with disinfectant or that decentralized canisters of disinfectant have to be used by users. The user must also manually fill the disinfectant to be used or determine a volume to be filled.

In addition, practice shows that those carrying out the work over-concentrate the disinfectant due to uncertainty about the amount to be added. On the one hand, this wastes chemicals and can possibly lead to an unauthorized pH value within the system. Alternatively, incorrect determination/calculation of the disinfectant quantity can lead to an under-concentration, which may result in incomplete disinfection. Although an overconcentration can lead to damage to the system, the risk of a potential underconcentration leads to the user creating an overconcentration.

### SUMMARY

The present disclosure is therefore based on the object of providing an improved process for the chemical disinfection of a reverse osmosis system, which overcomes the disadvantages of the prior art. In particular, this is intended to increase the accuracy of a desired initial target concentration of the disinfectant.

The method for the chemical disinfection of a reverse osmosis system, which comprises a supply tank, a ring line for connecting consumers, at least one membrane module, a feed line to the membrane module, at least one pressure pump for pumping liquid into the membrane module and a disinfectant container, the disinfectant being conveyed from the disinfectant container into the supply tank, comprises the steps of setting a target concentration of the disinfectant, determining the amount of disinfectant required on the basis of a target concentration of the disinfectant and discharging the amount of disinfectant required for the target concentration from the disinfectant container into the supply tank by a discharge device.

The present disclosure is based on the consideration that for reliable chemical disinfection of a reverse osmosis system, the specified target concentration of the disinfectant should be realized and maintained as precisely as possible. While too low a concentration of disinfectant jeopardizes the success of disinfection, too high a quantity can lead to component damage in the reverse osmosis system or the connected consumers due to the excessively high pH value.

The exact realization of the target concentration is not always guaranteed if the disinfectant is filled by hand by a user. It is also not possible to react to a change in the disinfectant mixture, i.e. the mixture of process water and disinfectant, in the chemical process.

As has now been recognized, an exact realization of the target concentration can be achieved by discharging the amount of disinfectant required to realize the target concentration from a disinfectant container provided for this purpose.

In contrast to methods known from the prior art, the actual amount of disinfectant required is calculated according to the present disclosure. This applies both to the initial filling and to the readjustment. A controlled disinfection with controlled pH value or concentration ratios is achieved.

The disadvantage of state-of-the-art processes, in which disinfectant is pumped into the circuit until a conductivity limit is exceeded, is that the concentration in the system is largely unclear/undefined, as the necessary parameters (total volume of liquid in the system) are unknown.

The term “disinfectant” refers to the agent or concentrate that is provided in the disinfectant container, while the term “disinfectant mixture” refers to the mixture of disinfectant and water.

The delivery/feeding of disinfectant from the disinfectant container into the supply tank is preferably automatic, but can also be carried out manually, i.e. by a user.

The disinfectant tank is advantageously a supply tank that can contain more disinfectant than is required for a planned disinfection of the reverse osmosis system with a specified target concentration of disinfectant. This ensures that sufficient disinfectant is available, even if consumers are disinfected at the same time and the required amount of disinfectant mixture increases during the process. The disinfectant container can also be designed as a canister. The disinfectant container or supply tank can be part of the reverse osmosis system or it can be an external canister that can be placed next to the reverse osmosis system and connected to it. Preferably, the disinfectant is pumped from a canister supplied with the system.

Advantageously, the method comprises the steps of monitoring and determining the current concentration of the disinfectant and, if the current concentration differs from the target concentration within a threshold range, feeding disinfectant and process inlet water into the supply tank in accordance with the target concentration. In this way, the system reacts to a changing total amount of disinfectant mixture over time and the amount of disinfectant mixture can be adjusted during the process. This makes it possible to also chemically disinfect connected consumers, in particular dialysis machines, during the chemical disinfection of the reverse osmosis system.

According to the present disclosure, monitoring and determining the current concentration of the disinfectant is carried out by determining the volume of liquid in the supply tank.

The volume of liquid is preferably determined by measuring the pressure with a pressure sensor. Here, a quantity of liquid is assigned to a corresponding change in pressure so that the change in the quantity of liquid can be inferred when a change in pressure is measured. Alternatively, or in combination, the liquid volume can also be determined mechanically (e.g. by means of a float), optically (e.g. by means of a light barrier) and/or electrically (e.g. by means of a conductivity measurement).

The volume of liquid is preferably determined by measuring the volumetric flow of at least one volumetric flow sensor. The volumetric flow sensor preferably measures the amount of liquid flowing from the reverse osmosis system into the ring line. In this way, liquid drawn from the consumers can be detected. In addition, the amount of disinfectant leaving the disinfectant tank can be determined using a volume flow sensor. In this way, it is possible to directly determine the amount of liquid drained from the disinfectant container and the supply tank by integrating the volume flows. If only one volume flow sensor is provided at the ring line inlet (as described here), the flow rate that flows back into the tank must be known and essentially continuous, as otherwise it is not possible to determine how much liquid leaves the circuit. In an alternative version, a second volumetric flow sensor can be attached to the ring line return in order to balance the two measured values against each other. The difference between the sensor values gives the consumed volume.

The current concentration of the disinfectant can preferably be determined using at least one substance-selective sensor. The substance concentration can be measured directly in this way and does not have to be calculated. This improves the accuracy of the concentration determination, as system properties that can falsify the result can be neglected.

The substance-selective sensor is preferably designed as an amperometric sensor. The amperometric sensor preferably reacts sensitively to hydrogen peroxide and/or peracetic acid and/or citric acid, which are usually the main components of modern disinfectants.

The current concentration of the disinfectant can preferably be determined by measuring the pH value. For this purpose, the molality of the hydrogen ions is determined from the measured pH value. A concentration can then be calculated using the molality and the known composition of the disinfectant.

In a first preferred embodiment, disinfectant and process input water are fed continuously into the supply tank. This ensures that the desired target concentration of the disinfectant is present in the system network, i.e. in the reverse osmosis system and the ring line and, if applicable, the consumers, as precisely as possible at all times.

In an alternative, preferred version, additional delivery takes place when the amount of liquid removed from the supply tank exceeds a volume threshold. This has the advantage that the measurement uncertainties and inertia of valves and pumps are easier to control.

In a preferred embodiment of the method, the delivery of disinfectant from the disinfectant container into the supply tank is carried out with a dosing pump, which is the rejection device.

In an alternative preferred version of the process, the disinfectant is conveyed from the disinfectant container into the supply tank by using gravity. For this purpose, in the assembled state or operating state of the reverse osmosis system for chemical disinfection, the disinfectant container is arranged higher than the maximum possible liquid column in the supply tank. For this purpose, a solenoid valve is advantageously arranged in a line between the disinfectant container and the supply tank, which is opened to feed disinfectant into the supply tank. The discharge device is realized in particular by the solenoid valve.

In both designs, an overflow container can be provided into which the disinfectant is fed before it is fed into the supply tank.

In a preferred embodiment of the process, the temperature of the disinfectant and/or the temperature of the disinfectant mixture, i.e. the mixture of disinfectant and water, is controlled. In addition to the purely chemical process, a heater and a temperature control unit can be integrated into the process for this purpose. In addition to the concentration, the temperature of the disinfectant and/or disinfectant mixture is also regulated. The heater or the heater can either be arranged in the reverse osmosis system, preferably in the supply tank, to control the temperature of the disinfectant or disinfectant concentrate and/or be connected externally to the reverse osmosis system/ring line to control the disinfectant mixture. The temperature is controlled via at least one temperature sensor. The temperature sensor is preferably located in the supply tank. Regulation is either carried out centrally, in particular by a control unit, or is delegated from the control unit to a heating control unit.

The reverse osmosis system has a supply tank, a ring line for connecting consumers, at least one membrane module, a feed line to the membrane module, at least one pressure pump for pumping liquid into the membrane module and a disinfectant tank. A control unit and means for carrying out the process described above are provided. The means can include, for example, valves and lines to enable the described flows of disinfectant and process water and which can be controlled by the control unit. The process can be implemented in software and/or hardware in the control unit.

Advantageously, the control unit has an interface for receiving and/or transmitting data. The control unit is advantageously capable of communicating with other devices using a data transmission protocol. Information such as target concentration and/or target temperature can be requested from consumers, in particular the dialysis machines, and set accordingly for the procedure. This data can also be sent back to the dialysis machines.

In this way, the control unit can either generate the lowest common concentration and/or temperature of all connected dialysis machines or enable staggered sampling. In the case of staggered sampling, similar profiles (temperature and/or concentration) are bundled and the corresponding devices are allowed or refused to sample the disinfectant mixture from the ring line.

The reverse osmosis system advantageously has at least one circulation pump for returning concentrate from the membrane module to the feed line.

The advantages of the present disclosure lie in particular in the fact that the method described can be used to disinfect a reverse osmosis system with a connected ring line, including the connected dialysis machines. In particular, the user has the advantage that the disinfectant no longer has to be dosed manually. In addition, the user can transfer the concentration and time specified by the disinfectant manufacturer as parameters and then no longer has to worry about any further action. This saves staff time and ensures a reproducible disinfection process in which no chemicals are wasted.

### BRIEF DESCRIPTION OF THE DRAWINGS

An embodiment of the present disclosure is explained in more detail with the aid of the following drawing figures.

FIG. 1 shows a reverse osmosis system in a first preferred embodiment;

FIG. 2 shows a cylindrical supply tank;

FIG. 3 shows a flow chart of a method in a preferred embodiment;

FIG. 4 shows a reverse osmosis system in a second preferred embodiment;

FIG. 5 shows a reverse osmosis system in a third preferred embodiment, and

FIG. 6 shows a reverse osmosis system in a fourth preferred embodiment.

Identical parts are marked with the same reference signs in all figures.

### DETAILED DESCRIPTION

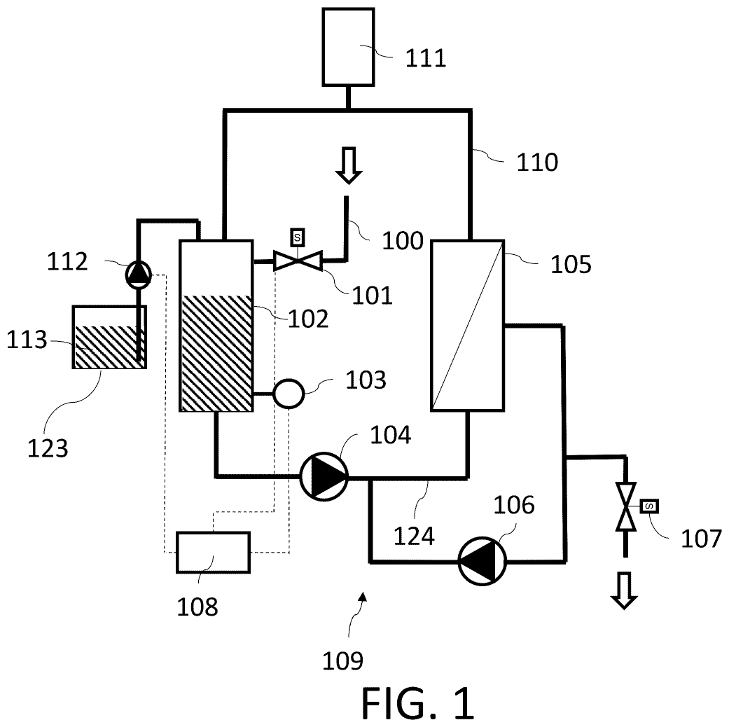

FIG. 1 shows a reverse osmosis system 109, which is connected to a ring line 110. Consumers, in this case dialysis machines 111, are connected to the ring line 110 at the consumption points.

During normal operation, the reverse osmosis system 109 produces permeate. For this purpose, process input water 100 is fed into a supply tank 102 via an open solenoid valve 101. The process input water 100 is fed via a pressure pump 104 through a feed line 124 into a membrane module 105. A first part of the volume flow, namely the concentrate, is either reintroduced into the process via a circulation pump 106 or discharged from the system via a solenoid valve 107. The other part of the volume flow, namely the permeate, is fed into the ring line 110. The dialysis machines 111 connected to the ring line 110 remove the permeate for dialysis treatment. The volume that is not removed is fed back into the supply tank 102.

The reverse osmosis system 109 has a discharge device which is designed as a metering/dosing pump 112 in order to pump disinfectant 113 from a disinfectant container 123 into the supply tank 102.

The control unit 108 is connected to the pressure sensor 103 on the signal input side and to the dosing pump 112 and the solenoid valve 101 on the signal output side.

In a chemical disinfection process, the control unit 108 continuously evaluates the pressure sensor 103. Based on the known geometry of the supply tank 102 and the evaluated pressure of the pressure sensor 103, the current tank volume of the supply tank 102 is calculated in liters. As a result, process input water 100 disinfectant 113 can be introduced into the supply tank 102 in the desired ratio.

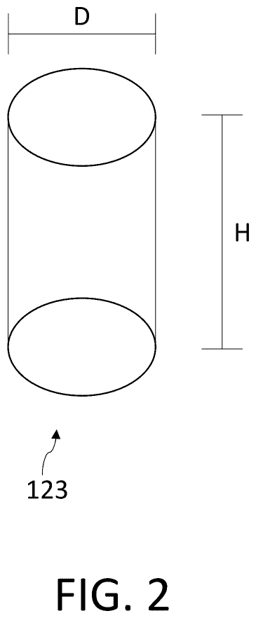

FIG. 2 schematically shows a supply tank 102 with a cylindrical cross-section, which has a diameter D and a height H, so that the volume of the supply tank 102 can be calculated from these variables in a known manner. This embodiment of the supply tank 102 is only exemplary. The actual design of the geometric shape of the supply tank 102 (round, square, etc.) is not decisive for the method.

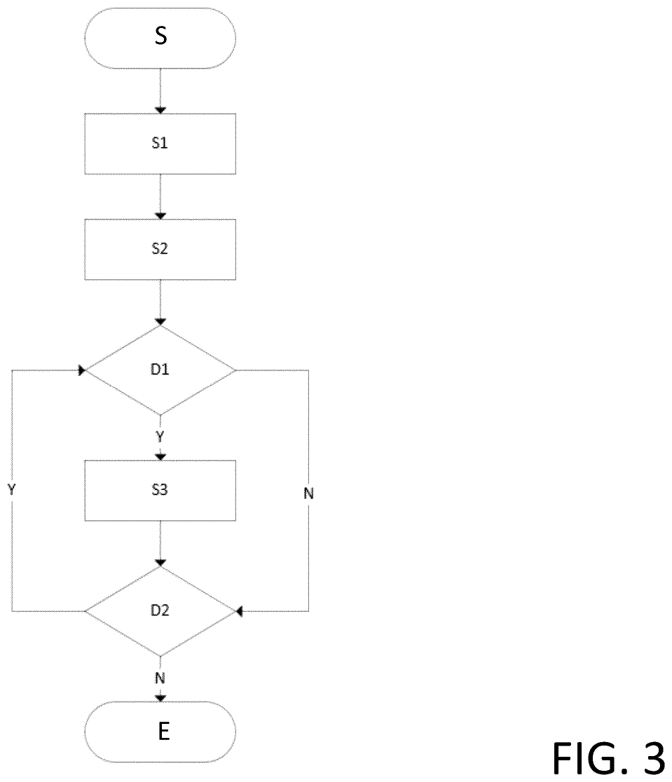

FIG. 3 shows a flow chart of a process for chemical disinfection in a preferred embodiment, which begins at a start S.

In a first sequence S1, a user starts the disinfection mode and transfers process data to the control unit 108 via a user interface (not shown), which is designed in particular as a GUI (Graphical User Interface). The process data or settings include at least the desired target concentration of the disinfectant. In addition to the desired target concentration information, the respective water volume of the reverse osmosis system 109 and the ring line 110 are required to carry out the process. The control unit 108 is designed or programmed in such a way that this information is stored in it. The volume of the ring line 110 can be specified either directly (in liters) or indirectly via the inner pipe diameter and the ring line length. Since these values tend to be parameters, as the system does not change spontaneously and/or frequently, these values can also be preset/stored by authorized personnel. The operating mode of reverse osmosis 109 for disinfection is also started in sequence S1.

After starting the operating mode and entering the process data or operating data in sequence S1, the required amount of disinfectant is calculated in sequence S2. The calculation is performed according to the following equation:

V_{(disinfectant)}[1]=(target concentration[%]/(1−target concentration[%]))*V_{total volume}[1].

Where V_{(disinfectant)} [1] is the required volume of disinfectant in liters, target concentration [%] is the target concentration of the disinfectant in percent, and V_{(total volume)} [1] is the system volume, i.e. the sum of the volumes of the reverse osmosis system 109 and the ring line 110 in liters. The symbol “*” represents multiplication.

As an example, the reverse osmosis system 109 with connected ring line 110 is to be disinfected at a concentration of 3%, whereby the total volume is 100 liters. This results in the volume of the disinfectant being (0.03/(1−0.03))*97 liters=3 liters.

The corresponding mixture of water and disinfectant is called a disinfectant mixture.

To start the disinfection process, the control unit 108 starts the pressure pump 104 and the circulation pump 106. A quantity of 3 liters of water is then discharged from the system network, i.e. the entire system consisting of the reverse osmosis system 109 and the ring line 110, by the control unit 108 opening the solenoid valve 107. This discharge is optional or does not take place if the tank volume is large enough and therefore no disinfectant would overflow. After the volume has been removed, the 3 liters of water are replaced by disinfectant 113, which is conveyed into the supply tank 102 by the dosing pump 112.

The volume of liquid in the supply tank 102 is determined with the aid of the pressure sensor 103. In the present case, the supply tank 102 shown in FIG. 2 has a diameter D of 0.30 m and a height H of 0.50 m.

The tank volume of the supply tank is calculated as follows:

π*(0.15 m)²*0.5 m=0.0353 m³=35.34 liters.

Assuming that a water column of 1 m corresponds to a pressure of approximately 98.07 mBar, each liter of water can be measured at 2,775 mBar. In the example above, the starting pressure must therefore lowered by 8,325 mBar then brought back to the starting value with disinfectant at in order to achieve the desired concentration.

After the initial concentration has been carried out in sequence S2, the tank volume of the disinfectant container 123 is monitored over the entire disinfection period. In a decision D1, it is checked whether the volume of disinfectant in the reservoir tank 102 decreases. This is the case when the at least one dialysis device 111 removes disinfectant. In this case, the method branches to a sequence S3.

In sequence S3, the disinfectant mixture is refilled. The removed disinfectant mixture is replaced. For this purpose, process input water 100 is added to the supply tank 102 via the solenoid valve 101 and disinfectant 113 is added in the corresponding quantity via the dosing pump 112 (see also sequence S2). This process does not have to be carried out continuously and can take place as soon as a minimum quantity, for example 5 liters, has been removed. This has the advantage that the measurement uncertainties and inertia of valves and pumps are easier to control. The supply tank 102 is only ever filled with one liquid at a time.

In a decision D2, it is checked whether the disinfection of the reverse osmosis system 109 is still being carried out. As long as disinfection is being carried out or is active, the above-mentioned concentration control is carried out and the process branches back to decision D1, otherwise it ends in an end E. As soon as the user or the control unit 108 changes the operating mode or the operating phase, the above-described concentration control is preferably carried out.

If it is determined in decision D1 that the volume of disinfectant in the disinfectant container 123 does not decrease, the method branches from there to decision D2.

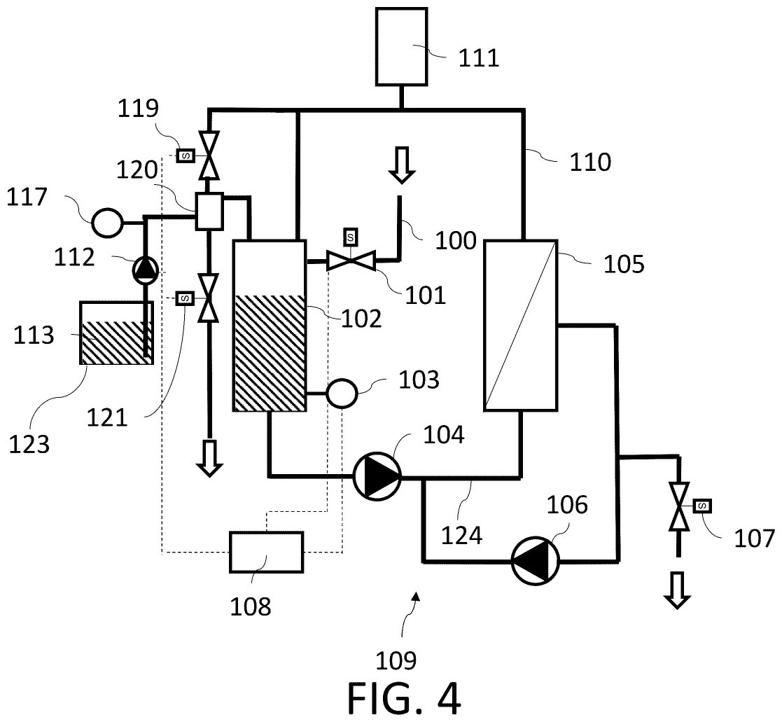

A reverse osmosis system 109 in a further preferred embodiment for the described process is shown in FIG. 4. The reverse osmosis system 109 has two solenoid valves 119 and 121 as well as an overflow tank 120, which is hydraulically connected between the dosing agent tank 123 and the supply tank 102. The solenoid valve 119 is hydraulically connected between the overflow tank 120 and the ring line 110. The solenoid valve 121 is connected to a line for discharging liquid from the overflow container 120 into a drain, so that the overflow container 120 can be emptied by opening the solenoid valve 121.

Solenoid valves 119 and 121 are never open or closed at the same time. One of the two solenoid valves 119, 121 is always open, while the corresponding other solenoid valve 121, 119 is closed. The respective switching state of the two solenoid valves 119, 121 is controlled by the control unit 108. If there is a need for disinfectant, the dosing pump 112 is activated so that it pumps disinfectant 113 into the overflow container 120. The volume of disinfectant is recorded by a volumetric flow sensor 117. Together with the permeate from the ring line 110, the overflow tank 120 is filled via the open solenoid valve 119 until the volume overflows into the supply tank 120. After disinfection is complete, the solenoid valve 119 is closed and the solenoid valve 121 is opened. The remaining disinfectant 113, which in the overflow tank 120, is directed into the drain. In this embodiment, the discharge device is provided by the dosing pump 112.

If the dosing pump 112 does not stop delivering disinfectant 113, this would not reach the supply tank 102 due to the open solenoid valve 121 and the higher overflow container 120.

To rinse out the overflow container 120, the solenoid valve 119 can be opened without switching on the dosing pump 112.

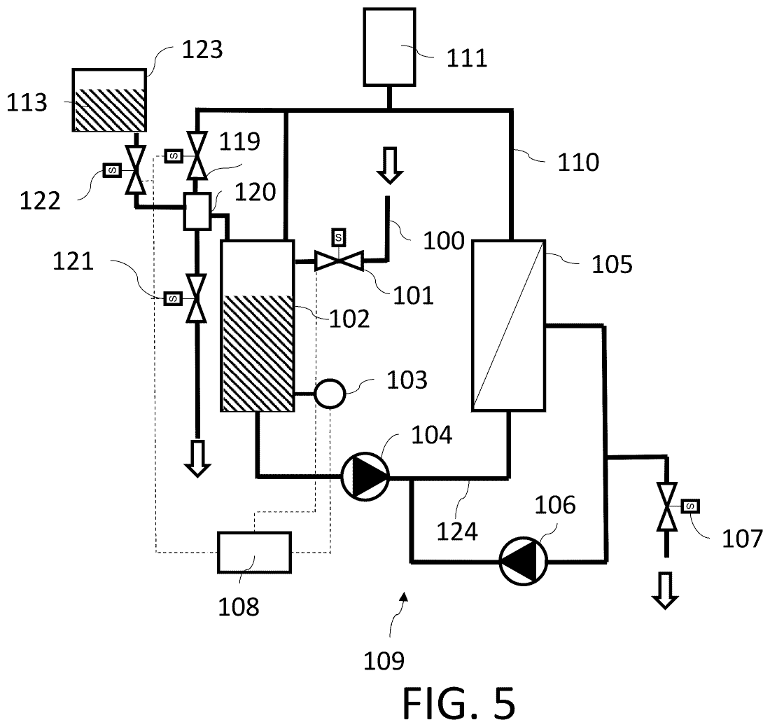

FIG. 5 shows a reverse osmosis system 109 in a further preferred embodiment for the process described, which does not have a dosing pump 112 for the disinfectant. Gravity is used to transport the disinfectant 113 into the supply tank 102. For this purpose, the disinfectant container 123 is mounted higher than the supply tank when the reverse osmosis system 109 is installed. The reverse osmosis system 109 has the two solenoid valves 119, 121 and the overflow tank 120. A solenoid valve 122 allows the disinfectant to flow out of the disinfectant tank 123. The discharge device is realized by the solenoid valve 122.

When the solenoid valve 122 is open, disinfectant first runs into the overflow tank 120 and then into the supply tank 102. In this reverse osmosis system 109, the solenoid valves 119, 121 remain closed for the duration of disinfection. After no more disinfectant is required, the overflow tank 120 is rinsed out together with the areas leading into the supply tank 102 by opening the solenoid valve 119. After rinsing is complete and until the next disinfection, the solenoid valve 119 is closed and the solenoid valve 121 is opened. the solenoid valve 122 is in the wrong position, the disinfectant can flow into the drain via the solenoid valve 121.

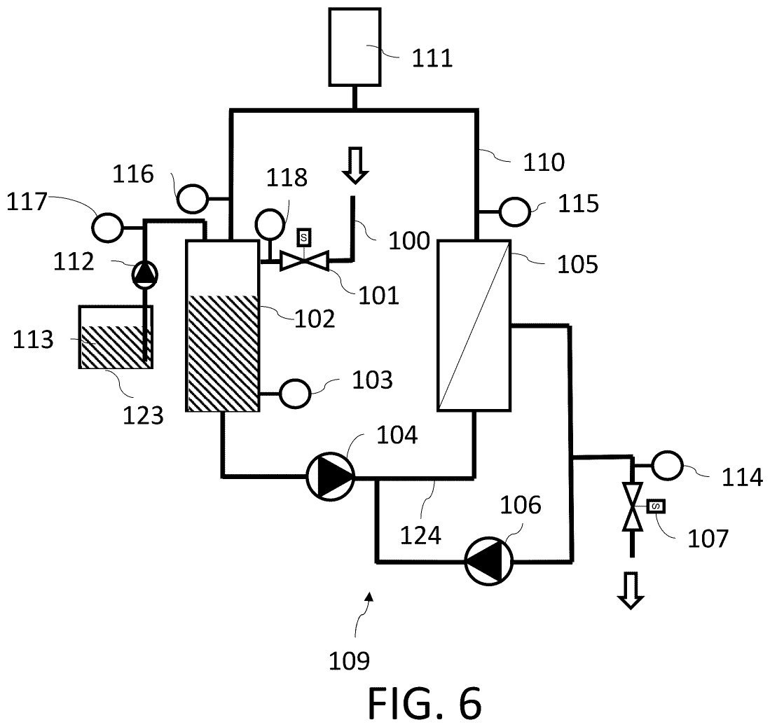

FIG. 6 illustrates another preferred embodiment of a reverse osmosis system 109 for the method. This reverse osmosis system 109 has two volumetric flow sensors 116, 117. The volumetric flow sensor 116 measures the volumetric flow from the supply tank 102 into the ring line 110. The volumetric flow sensor 117 measures the volumetric flow from the disinfectant tank 123 into the supply tank 102. By directly measuring the two volumetric flows, the quantity of the contents or the discharged contents in the supply tank or disinfectant 123 can be directly determined by temporal integration if the ring line return flow is known. The dosing pump 112 is the discharge device in this case.

### LIST OF REFERENCE SYMBOLS

100 process input water
101 solenoid valve
102 supply tank
103 pressure sensor
104 pressure pump
105 membrane module
106 circulation pump
107 solenoid valves
108 control unit
109 reverse osmosis system
110 ring line
111 dialysis machine
112 dosing pump
113 disinfectant
114 volume flow sensor
115 volume flow sensor
116 volume flow sensor
117 volume flow sensor
118 volume flow sensor
119 solenoid valve
120 overflow tank
121 solenoid valve
122 solenoid valve
123 disinfectant container
124 inlet pipe
D diameter
H height
S start
E end
S1 sequence
S2 sequence
S3 sequence
D1 decision
D2 decision

1. A method for chemical disinfection of a reverse osmosis system, the reverse osmosis system having a supply tank, a ring line for connecting consumers, at least one membrane module, a feed line to the at least one membrane module, at least one pressure pump for pumping liquid into the at least one membrane module and a disinfectant container, with disinfectant being conveyed from the disinfectant container into the supply tank, the method comprising the steps of:
setting a target concentration of the disinfectant;
determining an amount of disinfectant required based on the target concentration of the disinfectant; and
discharging the amount of disinfectant required for the target concentration from the disinfectant container into the supply tank through a discharge device.

2. The method according to claim 1, further comprising the steps of:
monitoring and determining a current concentration of the disinfectant; and
feeding disinfectant and process input water to the supply tank according to the target concentration when the current concentration of the disinfectant differs from the target concentration within a threshold range.

3. The method according to claim 2, wherein the step of monitoring and determining a current concentration of the disinfectant is performed by determining a liquid volume of the liquid in the supply tank.

4. The method according to claim 3, wherein determination of the liquid volume is carried out by a pressure measurement with a pressure sensor mechanically, optically and/or electrically.

5. The method according to claim 3, wherein the liquid volume is determined by a volume flow measurement of at least one volume flow sensor.

6. The method according to claim 2, further comprising the step of feeding disinfectant and process input water to the supply tank according to the target concentration when the current concentration of the disinfectant differs from the target concentration within a threshold range.

7. The method according to claim 6, wherein the step of feeding disinfectant and process input water to the supply tank is continuous.

8. The method according to claim 6, wherein feeding of disinfectant takes place when an amount of liquid removed from the supply tank exceeds a volume threshold.

9. The method according to claim 6, wherein feeding of disinfectant is carried out with a dosing pump as a discharge device.

10. The method according to claim 6, wherein the disinfectant is conveyed from the disinfectant container into the supply tank by gravity.

11. The method according to claim 2, wherein determination of the current concentration of the disinfectant is carried out with at least one substance-selective sensor.

12. The method according to claim 11, wherein the at least one substance-selective sensor is designed as an amperometric sensor.

13. The method according to claim 2, wherein determination of the current concentration of the disinfectant is carried out by a pH value measurement.

14. The method according to claim 1, wherein temperature of the disinfectant and/or a disinfectant mixture is controlled.

15. A reverse osmosis system comprising:
a control unit and means for carrying out the method according to claim 1;
a supply tank;
a ring line for connecting consumers;
at least one membrane module;
a feed line to the at least one membrane module;
at least one pressure pump for pumping liquid into the at least one membrane module; and
a disinfectant container.

16. The reverse osmosis system according to claim 15, wherein the control unit has an interface for receiving and/or transmitting data.
